(19) United States
(12) Patent Application Publication
(10) Pub. No.: US 2009/0326392 A1
(43) Pub. Date: Dec. 31, 2009

(54) METHOD AND SYSTEM FOR NON-INVASIVE BLOOD PRESSURE ESTIMATION

(75) Inventors: Sai Kolluri, Tampa, FL (US); Bruce Arnold Friedman, Tampa, FL (US); Richard Medero, Tampa, FL (US); Lawrence T. Hersh, Tampa, FL (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, SCHENECTADY, NY (US)

(21) Appl. No.: 12/146,572

(22) Filed: Jun. 26, 2008

(51) Int. Cl. A61B 5/02; A61B 19/00

(52) U.S. Cl. 600/494; 128/898; 600/485; 600/490; 600/493

(57) ABSTRACT

A method for non-invasively estimating blood pressure is disclosed herein. The method includes inflating a cuff and collecting first oscillation amplitude data at a first plurality of cuff pressure levels while inflating the cuff. The method includes identifying an artifact in the first oscillation amplitude data. The method includes identifying a specific cuff pressure level where the artifact occurs and deflating the cuff to the specific cuff pressure level. The method includes collecting second oscillation amplitude data at the specific cuff pressure level and estimating a blood pressure parameter based on both the first oscillation amplitude data and the second oscillation amplitude data. A non-invasive blood pressure system is also disclosed.

### BACKGROUND OF THE INVENTION

The subject matter disclosed herein relates to a method and system for non-invasive blood pressure estimation.

Human heart muscles periodically contract, forcing blood through the arteries. As a result of this pumping action, pressure pulses exist in these arteries and cause them to cyclically change volume. The minimum pressure for these pulses during a cardiac cycle is known as the diastolic pressure, and the peak pressure is known as the systolic pressure. A further pressure parameter, known as the mean arterial pressure, represents a time-weighted average of the blood pressure. Blood pressure parameters such as diastolic pressure, systolic pressure and mean arterial pressure are useful in monitoring the cardiovascular state of the patient.

A conventional method of measuring blood pressure is referred to as oscillometry. Typically, the measurement of blood pressure by oscillometry requires the inflation of a cuff to a cuff pressure level above the patient's systolic pressure to fully occlude the artery. Blood pressure is then determined by measuring an oscillation amplitude value at multiple cuff pressure levels during the deflation of the cuff. One problem with the conventional method is that the cuff may be inflated to an unnecessarily high cuff pressure level since the patient's systolic pressure is not known during the initial inflation of the cuff. This may lead to patient discomfort. Another problem with the conventional method is that if the initial cuff pressure level is too low, it may be necessary to inflate the cuff to a higher pressure level as part of one or more subsequent steps. An additional problem with the conventional method is that if one or more of the oscillation amplitude values contain an artifact, it may be necessary to reacquire the entire set of oscillation amplitude values as part of either an additional inflation process or during a deflation process. If it is necessary to reacquire the entire set of oscillation amplitude values, the blood pressure determination takes longer than necessary and may be a source of patient discomfort.

### BRIEF DESCRIPTION OF THE INVENTION

The above-mentioned shortcomings, disadvantages and problems are addressed herein which will be understood by reading and understanding the following specification.

In an embodiment, a method for non-invasively estimating blood pressure includes inflating a cuff and collecting first oscillation amplitude data at a first plurality of cuff pressure levels while inflating the cuff. The method includes identifying an artifact in the first oscillation amplitude data and identifying a specific cuff pressure level where the artifact occurs. The method includes deflating the cuff to the specific cuff pressure level and collecting second oscillation amplitude data at the specific cuff pressure level. The method also includes estimating a blood pressure parameter based on both the first oscillation amplitude data and the second oscillation amplitude data.

In another embodiment, a method for non-invasively estimating blood pressure includes inflating a cuff and collecting first oscillation amplitude data at a first plurality of cuff pressure levels while inflating the cuff. The method includes analyzing the first oscillation amplitude data for an artifact and identifying a specific cuff pressure level where the artifact occurs if the artifact is found while analyzing the first oscillation amplitude data. The method includes deflating the cuff and collecting second oscillation amplitude data at the specific cuff pressure level if the artifact is found while analyzing the first oscillation amplitude data. The method also includes estimating a blood pressure parameter using both the first oscillation amplitude data and the second oscillation amplitude data if the artifact is found while analyzing the first oscillation amplitude data.

In another embodiment, a system for non-invasively estimating a blood pressure parameter includes a cuff and a transducer attached to the cuff. The transducer is configured to acquire oscillation amplitude data. The system also includes a controller attached to the cuff. The controller is configured to collect first oscillation amplitude data from the transducer while inflating the cuff and to analyze the first oscillation amplitude data to identify an artifact. If the artifact is identified, the controller is further configured to identify a specific cuff pressure level of the artifact and to deflate the cuff in order to collect second oscillation amplitude data at the specific cuff pressure level. And, the controller is further configured to estimate a blood pressure parameter based on both the first oscillation amplitude data and the second oscillation amplitude data if the artifact is identified.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a non-invasive blood pressure system in accordance with an embodiment; and

FIG. 2 is a flow chart illustrating a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

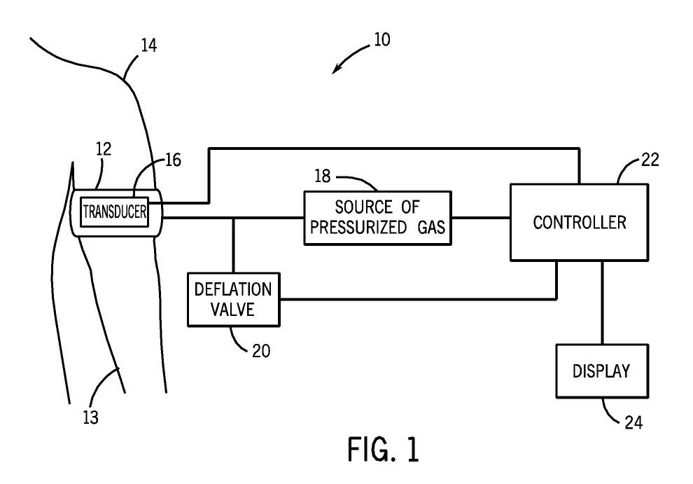

Referring to FIG. 1, a schematic representation of a non-invasive blood pressure (NIBP) system 10 is shown in accordance with an embodiment. The NIBP system 10 includes a cuff 12 wrapped about an arm 13 of a patient 14. The cuff 12 comprises one or more inflatable bladders (not shown) that can be selectively filled with air. While the cuff 12 is depicted around the arm 13 of the patient 14 in this embodiment, it should be appreciated that the cuff 12 could also be disposed about a leg or any other limb.

A transducer 16 is attached to the cuff 12 and configured to obtain a cuff pressure signal. The cuff pressure signal is used to determine measurements of a cuff pressure level and an oscillation amplitude. For the purposes of this disclosure, the “cuff pressure level” is defined to include a lower frequency portion of the cuff pressure signal, while the “oscillation amplitude” is defined to include the amplitude of a higher frequency portion of the cuff pressure signal that varies with the expansion and contraction of the patient's arteries. Both cuff pressure level and oscillation amplitude are well-known values in the oscillometric field.

A source of pressurized gas 18 is connected to the cuff 12 in a manner that allows gas to travel into the cuff 12 to increase the cuff pressure level. A deflation valve 20 is also connected to the cuff 12 and the deflation valve 20 functions to selectively lower the cuff pressure level. A controller 22 is operatively connected to the transducer 16, the source of pressurized gas 18, the deflation valve 20, and a display 24. The controller 22 is configured to regulate the source of pressurized gas 18 and the deflation valve 20 in order to attain a desired cuff pressure level. The controller 22 is also configured to receive the cuff pressure signal from the transducer 16. The display 24 is attached to the controller 22 and is adapted to display a blood pressure parameter as will be discussed in detail hereinafter.

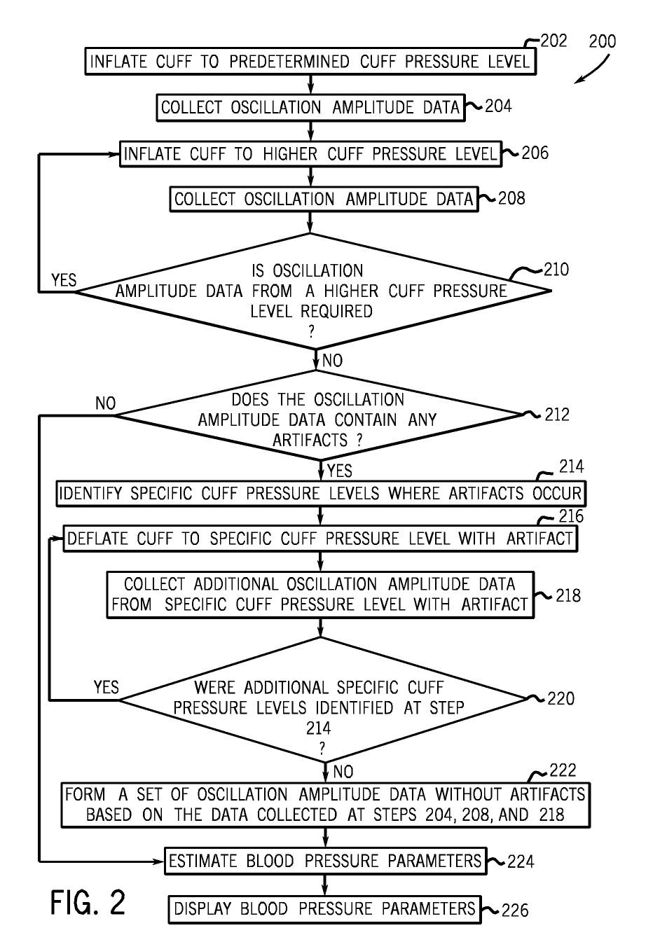

FIG. 2 is a flow chart illustrating a method 200 in accordance with an embodiment. The individual blocks 202-226 of the flow chart represent steps that may be performed in accordance with the method 200. The technical effect of the method 200 is the estimation of a blood pressure parameter based on oscillation amplitude data.

Referring to FIGS. 1 and 2, at step 202, the cuff 12 is inflated to a predetermined cuff pressure level. The controller 22 controls the source of pressurized gas 18 and the deflation valve 20 in a manner adapted to bring the cuff pressure level to the predetermined cuff pressure level. The predetermined cuff pressure level may be based on data from a previous estimation of the patient's blood pressure, it may be based on empirical data, or it may be manually set by an operator.

At step 204, oscillation amplitude data is collected at the predetermined cuff pressure level of step 202. For the purposes of this disclosure, it should be understood that oscillation amplitude data may comprise an oscillation amplitude value or a plurality of oscillation amplitude values. According to an embodiment, two oscillation amplitude values are collected at step 204.

At step 206, the cuff 12 is inflated to a higher cuff pressure level. At step 208, oscillation amplitude data is collected at the higher cuff pressure level of step 206. At step 210, after the oscillation amplitude data has been collected, the controller 22 determines if oscillation amplitude data from a higher cuff pressure level is required. This determination may be made by evaluating the oscillation amplitude data collected at steps 204 and 208 in order to see if a blood pressure parameter may be estimated. It should be appreciated by those skilled in the art that additional methods of determining if oscillation amplitude data from a higher cuff pressure level is required may also be employed at step 210.

If oscillation amplitude data from a higher cuff pressure level is required at step 210, the method 200 returns back to step 206, where the cuff 12 is inflated to a higher cuff pressure level. According to an embodiment, two oscillation amplitude values are collected from each cuff pressure level as the method 200 iteratively cycles through steps 206 through 210. It should be appreciated by those skilled in the art that the cuff pressure level may be increased in either a stepwise manner or in a continuous manner as the method 200 cycles through steps 206-210. For the purposes of this disclosure, “increased in a stepwise manner” is defined to include an inflation process where the cuff pressure level is increased in steps and where the cuff pressure level is maintained at a generally constant value at times when the oscillation amplitude data is collected. For the purposes of this disclosure, “increased in a continuous manner” is defined to include an inflation process where the cuff pressure level is continuously increased while oscillation amplitude data is collected. It should be appreciated by those skilled in the art that “increased in a continuous manner” includes methods employing both a generally constant rate of inflation of the cuff 12 and a variable rate of inflation of the cuff 12. It should also be appreciated that it may be possible to adaptively change the rate of inflation and/or the size of the steps between cuff pressure levels depending upon the oscillation amplitude data collected during steps 202-210 or the cuff pressure levels from which the oscillation amplitude data was collected during steps 202-210.

By collecting data while increasing the cuff pressure level according to steps 206-210, it is also possible to ensure that the cuff 12 is not inflated to an unnecessarily high cuff pressure level. For example, if the NIBP system 10 is no longer obtaining oscillation amplitude values because the patient's 14 artery is fully occluded, it may not be necessary to inflate the cuff 12 to a higher cuff pressure level.

If oscillation amplitude data from a higher cuff pressure level is not required at step 210, the method 200 proceeds to step 212. At step 212, the controller 22 determines if the oscillation amplitude data collected at steps 204 and 208 contains any artifacts. For example, according to an embodiment, determining if the oscillation amplitude data contains an artifact at step 212 may comprise analyzing the shape of a signal representing an individual oscillation. According to another embodiment, determining if the oscillation amplitude data contains an artifact at step 212 may comprise comparing an oscillation amplitude value to a neighboring oscillation amplitude value. According to another embodiment, determining if the oscillation amplitude data contains an artifact at step 212 may comprise analyzing an oscillation envelope for irregular oscillation amplitude values. For example, an embodiment may identify irregular oscillation amplitude values by identifying a change in the higher frequency portion of the cuff pressure signal corresponding to an individual oscillation. Additionally, an embodiment may identify irregular oscillation amplitude values by identifying a change in the lower frequency portion of the cuff pressure signal. It should be understood that different techniques for identifying artifacts in the oscillation amplitude data may be employed in additional embodiments.

If the oscillation amplitude data does not contain any artifacts at step 212, the method 200 proceeds to step 224 where a plurality of blood pressure parameters are estimated. The estimation of the blood pressure parameters at step 224 will be described in accordance with an embodiment hereinafter. Once the blood pressure parameters have been estimated at step 224, the blood pressure parameters are displayed according to step 226. According to an embodiment, only a single blood pressure parameter may be estimated and displayed.

Still referring to FIGS. 1 and 2, if the oscillation amplitude data is found to contain an artifact at step 212, the method proceeds to step 214. At step 214, a specific cuff pressure level where the artifact occurs is identified by the controller 22. Also, if artifacts are detected at multiple cuff pressure levels at step 212, then more than one cuff pressure level is identified at step 214.

According to another embodiment, the controller 22 may check the oscillation amplitude data for artifacts as the oscillation amplitude data is being collected. For example, after the oscillation amplitude data is collected for a given cuff pressure level, the controller 22 may check to see if the oscillation amplitude data contains an artifact. If the oscillation amplitude data contains an artifact, the controller 22 would store the specific cuff pressure level of the artifact.

At step 216, the controller 22 activates the deflation valve 20 and deflates the cuff 12 to the highest of the specific cuff pressure levels identified at step 214. At step 218, additional oscillation amplitude data is collected at the specific cuff pressure level. At step 220, the controller 22 determines if additional specific cuff pressure levels were identified at step 214. If additional specific cuff pressure levels were identified, then the method 200 returns to step 216 and the cuff is deflated to the next lower specific cuff pressure level identified at step 214. The method 200 iteratively cycles through steps 216 through 220 until additional oscillation amplitude data has been acquired at all of the specific cuff pressure levels identified at step 214.

According to an embodiment, the deflation of the cuff 12 to the specific cuff pressure level with the artifact at step 216 may comprise a multiple-step process. For example, the controller 22 may actuate the deflation valve 20 so that the cuff pressure is reduced at a first deflation rate until the cuff pressure level in the cuff 12 reaches a target cuff pressure level above the specific cuff pressure level identified at step 214. Then, the controller 22 may partially close the deflation valve 20 so that the cuff pressure level is reduced at a second deflation rate from the target cuff pressure level until the specific cuff pressure level identified at step 214 is reached. According to an exemplary embodiment, the target cuff pressure level may be selected to be within 10 mm of Hg of the specific cuff pressure level. According to an embodiment, the controller 22 may actuate the deflation valve 20 so that the cuff pressure level is reduced at the second deflation rate until the cuff pressure level is lower than the specific cuff pressure level identified at step 214. It should be understood that embodiments may collect the additional oscillation amplitude data while the cuff pressure level is being reduced at the second deflation rate, or embodiments may collect the additional oscillation amplitude data while the cuff pressure level is held generally constant.

Still referring to FIG. 1 and FIG. 2, at step 222, the controller 22 forms a set of oscillation amplitude data without artifacts by combining the oscillation amplitude data without artifacts from steps 204 and 208 with the oscillation amplitude data from step 218. For example, if additional oscillation amplitude data is collected at one specific cuff pressure level during step 218, the additional oscillation amplitude data from step 218 replaces or appends the oscillation amplitude data containing the artifact that had been collected at either step 204 or 208.

At step 224, the controller 22 uses the set of oscillation amplitude data without artifacts to estimate a plurality of blood pressure parameters. Examples of blood pressure parameters that may be estimated by the controller 22 include a mean arterial pressure, a diastolic pressure, and a systolic pressure. It should be understood that additional embodiments may estimate additional blood pressure parameters.

According to an exemplary embodiment, a curve may be fit to the oscillation amplitude data at step 224. A “best fit” algorithm may be implemented in order to ensure that the curve fits the oscillation amplitude data as accurately as possible. Once the curve has been fit to the oscillation amplitude data, the curve may be implemented in order to determine an estimate of a mean arterial pressure of the patient 14. The mean arterial pressure may be estimated by implementing the curve to find the cuff pressure level where the curve reaches a maximum oscillation amplitude. Once an estimate of the mean arterial pressure has been made, estimates of the diastolic pressure and the systolic pressure may be made based on well-know relationships of the diastolic pressure compared to the mean arterial pressure and the systolic pressure compared to the mean arterial pressure. According to an exemplary embodiment, the diastolic pressure may be estimated by finding the cuff pressure level below the mean arterial pressure where the ratio of the oscillation amplitude at the diastolic pressure to the oscillation amplitude at the mean arterial pressure equals a first established value, typically chosen to be between 0.4 and 1.0. According to an exemplary embodiment, the systolic pressure may be estimated by finding the cuff pressure level above the mean arterial pressure where the ratio of the oscillation amplitude at the systolic pressure to the oscillation amplitude at the mean arterial pressure equals a second established value, typically chosen to be between 0.4 and 1.0. While the mean arterial pressure, the systolic pressure, and the diastolic pressure are examples of blood pressure parameters, it should be understood that it would be possible to use the curve to estimate other blood pressure parameters as well.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method for non-invasively estimating blood pressure comprising:
inflating a cuff;
collecting first oscillation amplitude data at a first plurality of cuff pressure levels while said inflating the cuff;
identifying an artifact in the first oscillation amplitude data;
identifying a specific cuff pressure level where the artifact occurs;
deflating the cuff to the specific cuff pressure level;
collecting second oscillation amplitude data at the specific cuff pressure level; and
estimating a blood pressure parameter based on both the first oscillation amplitude data and the second oscillation amplitude data.

2. The method of claim 1, wherein said inflating the cuff comprises inflating the cuff in a continuous manner.

3. The method of claim 1, wherein said inflating the cuff comprises inflating the cuff in a stepwise manner.

4. The method of claim 1, wherein said estimating the blood pressure parameter comprises fitting a curve to both the first oscillation amplitude data and the second oscillation amplitude data.

5. The method of claim 1, wherein said identifying the artifact comprises analyzing a shape of an oscillation.

6. The method of claim 1, wherein said identifying the artifact comprises analyzing a shape of an oscillation envelope.

7. The method of claim 1, wherein said deflating the cuff comprises deflating the cuff to a target cuff pressure level above the specific cuff pressure level at a first deflation rate and then deflating the cuff to the specific cuff pressure level at a second deflation rate, wherein the first deflation rate is greater than the second deflation rate.

8. The method of claim 7, wherein said deflating the cuff to the target cuff pressure level comprises deflating the cuff to the target cuff pressure level that is within 10 mm of Hg of the specific cuff pressure level.

9. The method of claim 7, wherein said deflating the cuff to the target cuff pressure level comprises deflating the cuff to the target cuff pressure level that is within 5 mm of Hg of the specific cuff pressure level.

10. A method for non-invasively estimating blood pressure comprising:
inflating a cuff;
collecting first oscillation amplitude data at a first plurality of cuff pressure levels while said inflating the cuff;
analyzing the first oscillation amplitude data for an artifact;
identifying a specific cuff pressure level where the artifact occurs if the artifact is found during said analyzing the first oscillation amplitude data;
deflating the cuff and collecting second oscillation amplitude data at the specific cuff pressure level if the artifact is found during said analyzing the first oscillation amplitude data; and
estimating a blood pressure parameter using both the first oscillation amplitude data and the second oscillation amplitude data if the artifact is found during said analyzing the first oscillation amplitude data.

11. The method of claim 10, wherein said deflating the cuff comprises deflating the cuff to a target cuff pressure level above the specific cuff pressure level at a first deflation rate and then deflating the cuff to the specific cuff pressure level at a second deflation rate, wherein the first deflation rate is greater than the second deflation rate.

12. The method of claim 10 wherein said deflating the cuff comprises deflating the cuff in a continuous manner.

13. The method of claim 10, wherein said inflating the cuff comprises inflating the cuff in a stepwise manner.

14. The method of claim 10, wherein said estimating the blood pressure parameter comprises fitting a curve to both the first oscillation amplitude data and the second oscillation amplitude data.

15. The method of claim 10, wherein said analyzing the first oscillation amplitude data for the artifact comprises analyzing a shape of an oscillation.

16. The method of claim 10, wherein said analyzing the first oscillation amplitude data for the artifact comprises analyzing a shape of an oscillation envelope.

17. The method of claim 10, wherein said analyzing the first oscillation amplitude data for the artifact occurs during said collecting the first oscillation amplitude data.

18. A system for non-invasively estimating a blood pressure parameter comprising:
a cuff;
a transducer attached to the cuff, the transducer configured to acquire oscillation amplitude data; and
a controller attached to the cuff;
wherein the controller is configured to collect first oscillation amplitude data from the transducer while inflating the cuff and to analyze the first oscillation amplitude data to identify an artifact;
wherein if the artifact is identified, the controller is further configured to identify a specific cuff pressure level of the artifact and to deflate the cuff in order to collect second oscillation amplitude data at the specific cuff pressure level;
wherein if the artifact is identified, the controller is further configured to estimate a blood pressure parameter based on both the first oscillation amplitude data and the second oscillation amplitude data.

19. The system of claim 18, further comprising a display connected to the controller.

20. The system of claim 18, wherein the controller is further configured to estimate the blood pressure parameter by fitting both the first oscillation amplitude data and the second oscillation amplitude data to a curve.
